(19) United States
(12) Patent Application Publication
(10) Pub. No.: US 2010/0004344 A1
(43) Pub. Date: Jan. 7, 2010

(54) ANTI-OXIDANT DIETARY COMPOSITION CONTAINING FRUITS AND VEGETABLES, METHOD FOR PREPARING THE SAME AND USE OF THE COMPOSITION

(75) Inventor: Constantin Dallas, Beziers (FR)

Correspondence Address: THE NATH LAW GROUP, 112 South West Street, Alexandria, VA, 22314, US

(73) Assignee: NBC NUTRACEUTIC BUSINESS CONSULTING, Beziersfr (FR)

(21) Appl. No.: 12/448,936

(22) PCT Filed: Jan. 17, 2007
(86) PCT No.: PCT/FR2007/050657

§ 371 Date: Jul. 29, 2009

(51) Int. Cl. A61K 31/05; A23L 1/302; A23L 1/30; A23L 1/216

(52) U.S. Cl. 514/734; 426/541; 426/72; 426/96

(57) ABSTRACT

The invention relates to a dietary composition containing at least polyphenols and cartenoids. It is advantageously obtained from a mixture of vegetal species containing at least red and/or white grapes (Vitis vinfera), blueberries (Vaccinium myrtillus), tomatoes (Solanum lycopersicum), carrots (Daucus carrota), and green tea (Camelia sinensis). The invention also relates to a method for preparing the composition and the use of the composition as a food complement and/or for enriching food products in order to reduce the global cholesterol level, in particular the level of cholesterol related to low density proteins (LDL), and/or the atherogenic index, and/or for increasing the antioxidant capacity of blood plasma and/or for reducing the amount of free radicals in the organism. It is advantageously delivered following a dosage of between 10 to 25 mg, preferably of about 21.5 mg of dietary composition per kilogram of body mass per day.

### BACKGROUND OF THE INVENTION

### (1) Field of the Invention

The invention relates to an antioxidant composition for dietary use, in particular obtained from fruits and vegetables.

The invention also relates to a method for obtaining such a composition as well as the use of such a composition.

The invention regards the field of food supplements.

In the field of diets is known the interest of an appropriate consumption of fresh fruits and vegetables for the benefits it provides to the organism.

It is thus considered that a balanced diet should include at least five different fruits and vegetables per day, corresponding to approximately 400 grams in weight of fresh fruits and vegetables.

Because of the constraints of modern life, in particular in the city, attaining such an objective is most often uncertain.

The object of this invention is to provide a solution for attaining this objective.

In addition, the dietary choices of modern consumption encourage most often a considerable intake of fats or lipids to the detriment of other nutritive elements that are by no means less essential, whereby the consequences may range from unreasonable weight gain to, for example, triggering cardiovascular diseases caused by the accumulation of lipids inside the arteries.

Thus, another object of this invention is to provide a solution for preventing the development of cardiovascular diseases.

It is also known that an unbalanced diet, leading to nutritional weaknesses and even deficiencies, is a source of oxidative stress for living organisms. This oxidative stress can also be associated to our life environment (pollution, tobacco, U.V., etc.) or result from these various factors (food and/or life environment) taken together. This oxidative stress is due to an excess of oxygenated reactive species, such as superoxide anions, hydrogen peroxide or hydroxyl radicals, in the cells of the organism with respect to said cells' capacities to control them.

This oxidative stress is also partially the cause of the effects of ageing, in particular of the skin.

Yet another object of the invention is to provide an anti-ageing solution.

The invention pretends to make up for these multiple disadvantages, which lead to or result from a misbalanced diet, by providing a food supplement specifically designed for its both antioxidant and targeted action for preventing risks of cardiovascular diseases.

To this end, the invention relates to a dietary composition, wherein the composition includes at least polyphenols and carotenoids.

Advantageously, the dietary composition according to the invention also includes vitamin C and B-type vitamins.

The content of polyphenols in the composition is at least 60% by weight, carotenoids 0.1 to 2% by weight, vitamin C 0.1 to 2% by weight and B-type vitamins 0.1 to 1.5% by weight.

The composition is advantageously obtained from a mixture of vegetal species including at least red and/or white grapes (Vitis vinifera), blueberries (Vaccinium myrtillus), tomatoes (Solanum lycopersicum), carrots (Daucus carota), and green tea (Camelia sinensis).

The dietary composition is advantageously in the form of a powder having antioxidant capacity corresponding to at least 5000 moles/gram of Trolox equivalents.

The invention also relates to a method for obtaining such a composition.

The invention also relates to the use of the composition as food supplement and/or for enriching foods.

The invention also relates to the use of the composition for its dietary administration, in particular according to a dosage between 10 and 25 mg, preferably approximately 21.5 mg, of the composition per kilogram of body mass per day.

The objectives and advantages of this invention will become clear from the following detailed description referring to several exemplary embodiments. The understanding of this description will be made easier when referring to the attached drawings.

### BRIEF DESCRIPTION OF THE DRAWINGS

FIGS. 1 through 3 present experimental results attesting the effectiveness of the composition according to the invention.

### DETAILED DESCRIPTION OF THE PREFERRED EMBODIMENTS

This invention regards the field of food supplements and refers, more specifically, to an antioxidant composition for dietary use, in particular obtained from fruits and vegetables.

The vegetal species for its obtainment have been chosen based on their nutritional content, beneficial for the health, so as to obtain a composition providing an appropriate dosage of active substances acting in synergy.

In particular, and according to a particular embodiment, the composition according to the invention puts to good use a synergy between the effects of polyphenols and carotenoids it contains.

Advantageously, and according to a preferred embodiment, the synergy is increased by adding vitamin C and several types of vitamins B administered as a single intake.

Polyphenols constitute a group of chemical substances found in plants, and having antioxidant properties. The composition according to the invention contains more precisely procyanidins, flavanols and anthocyanins.

Carotenoids are essentially present in the form of lycopene and/or beta-carotene.

Table 1 shows the dosage of these different essential active substances in a preferred embodiment of the composition according to the invention.

More specifically, the dietary composition according to the invention includes, by weight, at least 60% of polyphenols and 0.1 to 2% of carotenoids.

Polyphenols are more specifically represented by procyanidins, flavanols and anthocyanins.

Advantageously, the composition includes more specifically, by weight, 1 to 30% of procyanidins, 5 to 50% of flavanols and 0.1 to 10% of anthocyanins.

Carotenoids have a content of lycopene and/or beta-carotene equivalent to 0.1 to 1% by weight of the final composition for each molecule.

If need be, vitamin C is present, by weight, in the range of 0.1 to 2% and vitamins B in the range of 0.1 to 1.5%.

Type B vitamins are part of the group comprised of vitamin B1, vitamin B6 and vitamin PP, separately or in any combination between the constituents of this group, and with contents in the composition of approximately 0.1 to 0.5% for vitamins B1 and B6, and approximately 0.1 to 1% for vitamin PP.

The percentages are calculated based on the analysis of a composition sample using various methods. Total polyphenols dosing is made by means of Ultraviolet spectrophotometry at 280 nm, and anthocyanins by means of the same technique at 520 nm. Total carotenoids dosing is also made through Ultraviolet spectrophotometry. Procyanidins, flavanols, vitamin C and vitamins B dosing is made by High Performance Liquid Chromatography (HPLC).

An optimized method that we will describe subsequently permits to obtain the dietary composition according to the invention, which is in the end in the form of a powder, standardized in antioxidant capacity. Advantageously, 1 gram of the composition according to the invention permits to deliver at least 5000 ORAC values (ORAC=Oxygen Radical Absorbance Capacity), this means that the final powder composition contains at least 5000 μmoles/gram of Trolox equivalents, which is also a conventional measure of a substance's oxidizing potential.

This oxidizing potential calibrated at 5000 ORAC values per gram corresponds to well-considered specifications for the elaboration of the composition and its obtainment method according to the invention. Actually, results of studies on the oxidizing potential of fruits and vegetables have shown that five fruits and vegetables of a total weight of 400 grams contain on average approximately 4000 ORAC values. A composition delivering 5000 ORAC values per gram of composition permits, by administering 0.8 grams of composition, to attain the 4000 ORAC values contained in a consumption of 400 grams of fresh fruits and vegetables. Likewise, the administration of 1.6 grams of composition according to the invention will permit to reach the 8000 ORAC values that 800 grams of fruits and vegetables contain, generally attained by the consumption of 10 fruits and vegetables per day.

Surprisingly, experiments have confirmed that the active substances dosage mentioned above is particularly effective for matching the dietary objectives aimed at, i.e. an anti-ageing effect and reduction of risks of cardiovascular diseases.

In order to ensure a better comprehension of these experiments, it is necessary to set out beforehand the biological factors at work in certain cardiovascular diseases such as atheroma, which is one of the predominant etiologies of the majority of cardiovascular ailments.

An atheroma corresponds to a modification of the intima of the large- and medium-size arteries as a result of the segmental accumulation of lipids, complex carbohydrates, blood and blood products, adipose tissues and calcareous deposits.

In particular, it is known that the development of an atheroma is initiated by the oxidation of low density lipoproteins (LDL). This oxidation is, in its turn, favored by a considerable oxidative stress, whether its origin is dietary and/or environmental.

In this respect, low density lipoproteins (LDL) transport cholesterol from secretion spots toward the cells of the organism.

As a matter of fact, since cholesterol is a hydrophobic compound, it is not soluble in blood and its transport is ensured by lipoproteins, which it is fixed to.

Considerable levels of low density lipoproteins (LDL) generally lead to the depositing of cholesterol on the walls of the arteries in the form of atheroma plaque, which increases the risk of cardiovascular diseases and has earned them the name <<bad cholesterol>>.

High density lipoproteins (HDL), in turn, take cholesterol away from arteries and extrahepatic tissues, transporting it to the liver where it is degraded. These lipoproteins are generally referred to as <<good>> cholesterol.

The causes of oxidative stress favoring the atheroma are not well known but recent works have shown that one of the major sources generating oxygenated reactive species, which cause oxidative stress, is formed by the activity of an enzyme, NAD(P)H oxidase.

This enzyme associated to the membranes is comprised of 5 sub-units, and catalyses oxygen reduction, by using NADP or NADPH as electron source. NAD(P)H oxidase generates significant quantities of superoxide radicals and an association between its enzymatic activity and the factors of clinical risks of atheroma have been proven. Azumi et al. (Expression of NADH/NADPH oxidase p22phox in human coronary arteries. Circulation 1999, 100, 1494-8) have shown that the severity of lesions appearing in the atheroma was correlated with the overexpression of sub-unit p22phox, a constituent of NAD(P)H oxidase, in coronary arteries.

As a matter of fact, it seems that an amplifying cycle is initiated in case of an atheroma, which constitutes a pathological situation, where membrane units of NAD(P)H oxidase, such as unit p22phox, are overexpressed. NAD(P)H oxidase has thus a recognized role in atheroma pathogenesis.

Turning back to the experiments, they have been conducted on male Golden Hamsters, also called Syrian Hamsters, having a weight varying between 85 and 95 grams.

The choice of hamsters for carrying out these experiments is justified by the fact that hamsters' distribution of plastmatic lipoproteins is similar to the one found in humans and that the major carriers of plastmatic cholesterol are low density lipoproteins (LDL).

A number of these animals have been subjected to a qualified atherogenic diet, i.e. designed to favor the appearance of atheroma, since it is rich in cholesterol and in fats. In order to induce a peroxidative stress, this diet has also been made deficient in vitamins C and E as well as in selenium.

Also, a composition according to the invention diluted in water was administered, according to procedures specified further in the description of the experiments, to a number of these animals. An analysis of the composition used in these experiments was made beforehand and is summarized in Table 2.

The conducted experiments can be summarized in three experiments revealing the physiological effects of an administration of the composition according to the invention.

### Experiment 1

A first experiment, the results of which are presented in Table 4, was aimed at the detection of the effects of an administration of the composition according to the invention on plasmatic concentrations of lipids and the antioxidant capacity of blood plasma in hamsters subjected to an atherogenic diet.

This experiment was conducted on two groups of 18 hamsters each, each group having an equivalent average in terms of body masses. Hamsters of each one of said two groups have been fed for 84 days based on an atherogenic diet.

This atherogenic diet consists in administering 200 g/kg of body mass of casein, 3 g/kg of L-methionine, 393 g/kg of corn starch, 154 g/kg of sucrose, 50 g/kg of cellulose, 150 g/kg of lard, 5 g/kg of cholesterol, a mixture of minerals amounting to 35 g/kg and a mixture of vitamins amounting to 10 mg/kg. Besides, the mixture of vitamins includes neither selenium nor vitamins E and C.

The hamsters have also been hydrated by force either by using tap water for the first group, referred to as control group, or using a composition according to the invention diluted in water for the second group, referred to as experimental group. The volume of administered solutions was adjusted daily to the weight of each animal, according to the volume rule of 7.14 mL per kilogram of body mass. The hamsters of the experimental group have received a dose of 21.4 mg per kilogram of body mass of the composition according to the invention, dissolved in a volume of water calculated according to the rule mentioned above.

This dose of 21.4 mg of composition per kilogram of body mass comes from a correlation made with a consumption in humans of approximately 800 grams of fresh fruits and vegetables per day. As seen previously, the ORAC value of this consumption is attained by the administration of 1.6 grams of composition according to the invention. Considering that the average human consumer weighs approximately 70 kg, one has managed to reproduce, in hamsters, the ingestion of approximately 10 fruits and vegetables per day, of a weight of approximately 800 grams, by administering them this dose of 21.4 mg of composition per kilogram of body mass daily.

At the end of this treatment, the level of total cholesterol and the level of cholesterol related to high density lipoproteins (HDL) was determined by using commercial enzymatic methods.

The antioxidant capacity of plasma was measured in Trolox equivalents, which is a quantitative measure of the general level of antioxidants in biological samples. The conventional technique is a colorimetric technique and shows the capacity of a sample to eliminate a colored cationic radical.

It should be noted that no difference was found between initial body masses, final body masses, and the quantity of food taken between the two groups, as summarized in Table 3.

As is evident from the results shown in Table 4, the administration of the composition according to the invention has permitted the reduction of the level of total plasma cholesterol by 15% and the level of cholesterol not related to high density lipoproteins (HDL), therefore essentially the level of cholesterol related to low density lipoproteins (LDL), by 33% with respect to the control group. The level of cholesterol related to high density lipoproteins (HDL) has not changed. Therefore, the atherogenic index, corresponding to the ratio between the total cholesterol and the cholesterol related to high density lipoproteins (HDL), was reduced by 12.3% in the hamsters that have received the antioxidant composition according to the invention.

In addition, the administration of the composition according to the invention has permitted to reinforce the antioxidant capacities of blood plasma, improving theses capacities by 10%.

To conclude, the administration of the composition according to the invention improves the lipid profile of blood plasma and reinforces its antioxidant capacities.

### Experiment 2

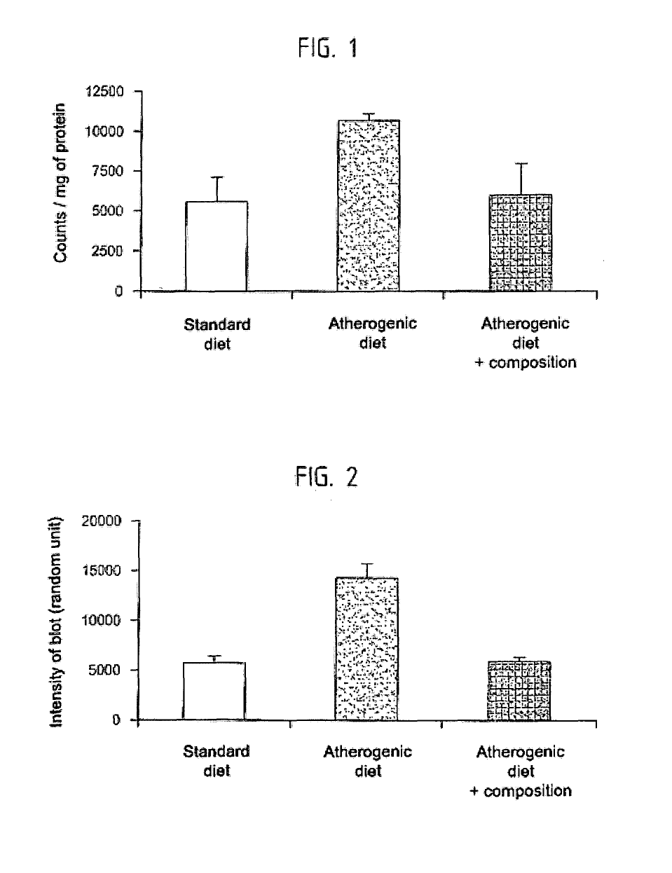

A second experiment, the results of which are shown in FIGS. 1 and 2, was aimed at the detection of the production of superoxide anion, oxidative stress agent produced predominantly by NAD(P)H oxidase, and the evolution of the expression of NAD(P)H oxidase during a treatment with the composition according to the invention.

This experiment was performed at the same time on hamsters fed with a standard diet, hamsters fed with an atherogenic diet and an administration by force of tap water, and hamsters fed with an atherogenic diet and an administration of water, which the composition according to the invention was added to.

The standard diet consists in administering 200 g/kg of body mass of casein, 3 g/kg of L-methionine, 447 g/kg of corn starch, 175 g/kg of sucrose, 50 g/kg of cellulose, 80 g/kg of vegetable oils, a mixture of minerals amounting to 35 g/kg and a mixture of vitamins amounting to 10 mg/kg.

The three groups of hamsters, each comprised of 6 individuals, have been fed for 84 days in the same conditions as those of experiment 1.

At the end of this treatment, the determination of the production of superoxide anion was assessed by a conventional technique for detecting the superoxide anion by means of chemiluminescence. The hamsters' left ventricles have been placed in a buffer solution containing 250 moles of lucigenin and the intensity of the resulting luminescence was measured with a luminometer.

The results of this operation, performed on the three groups containing 6 hamsters each, are shown by the graph of FIG. 1. This graph shows in the ordinate the measurement, average on all hamsters of a group, of chemiluminescence in counts per mg of protein, this value being the higher as the presence of superoxide anion is considerable. In the abscissa are shown the three groups of hamsters under observation.

Then one proceeded to extracting proteins contained in the frozen left ventricles of the same hamsters so as to proceed, conventionally, to the immunoblotting of the sub-unit p22phox, in order to measure the differences of expression of NAD(P)H oxidase in each of the 3 groups of hamsters.

The results shown on the graph of FIG. 2 are obtained based on an immunoblotting data processing software after acquisition of the image of the obtained gel, and show the relative presence of the sub-unit p22phox by a measurement of the intensity of the blots obtained on the gel. In the ordinate is shown the intensity of the blot corresponding to the sub-unit p22phox on the gel, in a random unit. In the abscissa are shown the three groups of hamsters under observation.

These results show that the production of superoxide anion (FIG. 1) and the expression of the sub-unit p22phox (FIG. 2) have respectively diminished by 45.5% and 59.1% in the hamsters that received the composition according to the invention, compared to the hamsters under atherogenic diet that did not receive it.

### Experiment 3

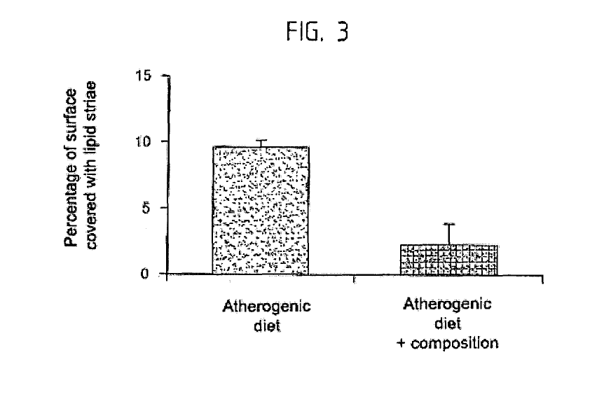

A third experiment, the results of which are shown in FIG. 3, was aimed at the visualization, at the level of the hamsters' aorta, of the extension of lipid striae.

As a matter of fact, the atheroma starts by lipid infiltrations, referred to as lipid striae, at the level of the intima, leading to thickening of said intima. Then there is a proliferation of smooth muscle cells and of connective tissue, leading to the formation of an unstable inflammatory plaque.

This experiment was carried out at the same time on hamsters fed with a standard diet, hamsters fed with an atherogenic diet and an administration by force of tap water, and hamsters fed with an atherogenic diet and an administration of water, which the composition according to the invention was added to.

The three groups of hamsters, each comprised of 12 individuals, have been fed for 84 days in the same conditions as those of experiments 1 and 2.

At the end of this treatment, the animals have been sacrificed, and after having collected their blood and extracted their liver, their aorta was perfused with a solution for the fixation of their vascularization and dissected between the sigmoid valves and 3-4 cm after the aortic arch. The extracted aortic portion was then cleaned, cut and opened longitudinally, then plunged into a fixing solution. After rinses, aortic segments were placed on a glass for microscopic preparation, with the endothelial side upwards, and mounted on a microscope. After photographing and digitizing, intima surfaces having lipid striae were expressed as a percentage of the total of the examined surfaces.

The graph of FIG. 3 shows in the ordinate these average percentages in each group of hamsters under observation, the extension of the lipid striae increasing with the value of said percentage. In the abscissa are shown the two groups of hamsters in which lipid striae have been observed.

It should be noted that no lipid stria was detected in the hamsters subjected to the standard diet. The average accumulation of aortic lipid striae was significantly reduced by 77% in the hamsters that received the composition according to the invention, compared to the hamsters under atherogenic diet that did not receive it.

This shows the beneficial effect of the composition according to the invention for an effective reduction of the macroscopic factors precursors of cardiovascular diseases.

In order to obtain the composition according to the invention, one proceeded to selecting fruits and vegetables the combination of which is particularly suited for obtaining said composition. Table 5 shows the necessary essential vegetal species, according to a particular embodiment, in which the composition is obtained from a mixture of grapes (red and white), blueberry, tomato, carrot and green tea. Advantageously, a percentage by weight fixing the representation of each vegetal species is respected.

Table 6 shows a preferred embodiment, based on a mixture of 22 fruits, vegetables and various other vegetal species.

Finally, the composition according to the invention is obtained thanks to a preferred method, the stages of which are retranscribed below.

In particular, at the level of cropping and transporting the raw materials as well as during the extraction stage, it is advisable to protect, as much as possible, the active substances, in particular the polyphenols, against oxidation. To this end, strategies such as working under inert atmosphere can be applied, in particular during the extraction.

A first stage consists in grinding the raw materials. Selected vegetal species are ground, individually or combined in different groups, in a grinder with one or more knives, at high speed. All alimentary parts of the vegetal species, namely fruits and vegetables, can be used, i.e. the skin, the core, the juice, the seeds or also the leaves. The knives are driven in rotation at a speed of 6,000 to 12,000 revolutions per minute and for a period of 2 to 5 minutes.

A second stage consists in extracting active substances from the ground materials by maceration. The extraction can be made with water preheated to a temperature of 50 to 90° C. or by using organic solvents, for example ethanol or ethyl acetate, that are compatible with the use in the food industry. Preferably, a double extraction is made by using a mixture of water and organic solvents in the proportion of 70 to 30% of water and 30 to 70% of organic solvents, respectively.

The extraction process is carried out in a stainless-steel tank and under inert atmosphere in order to avoid the oxidation of active molecules, at a temperature between 40 and 90° C. and being stirred for 5 to 20 hours.

Various parameters have an influence on maceration time, such as the concentration of active substances of the feedstock, the fineness of grinding obtained, the extraction temperature or the solvents used.

Other conventional operations could be carried out for such an extraction, such as successive macerations, extraction under reflux or under reduced pressure.

A third stage consists in centrifuging the macerate. After a rest without stirring at ambient temperature for 10 to 20 hours, the macerate is centrifuged so as to separate all solid particles thereof, and retrieve the liquid phase containing the extracted active substances.

A forth stage consists in concentrating the active substances. The liquid phase obtained is concentrated by distillation or evaporation. This stage lasts between 5 and 15 hours. The distillate or the re-solubilized evaporation residue, containing the extracted active substances, is then subjected to measurement of its ORAC value, according to conventional techniques.

From a qualitative point of view, the ORAC value of the required liquid phase at this stage should be comprised between 15,000 and 25,000 μmoles/gram of Trolox equivalents.

Additional purification stages can also be added. The concentrated extract can thus be subjected to other conventional purification operations such as filtration through cellulose membrane and/or discoloration.

A last stage consists in drying by atomization the concentrated extract resulting from the preceding operations. The latter is finally mixed with a matrix, preferably maltodextrine compatible with an alimentary use, and pulverized for drying by means of conventional techniques so as to obtain a fine powder, containing at least approximately 5,000 μmoles/gram of Trolox equivalents (ORAC value) and 50 to 90% of polyphenols.

Through the method according to the invention, it is thus possible to obtain a composition that advantageously solves the problem set forth, i.e. permitting to restore a daily dietary intake of fruits and vegetables, taking preventive actions against cardiovascular diseases and fighting the general effects of ageing through its antioxidant capacity.

Advantageously, the powder obtained at the end of the method according to the invention is water-soluble.

The invention also relates to the use of the obtained composition as food supplement and/or for enriching foods.

In a non-exhaustive way, the obtained powder can be used, on its own or with additional additives, in the dosage form of free powder, encapsulated powder in order to avoid, in particular, its oxidation, for example in capsules, powder compressed into tablets or granules of all shapes, or also powder put into sachets.

The powder obtained can also be used, on its own or with additional additives, for example dissolved in an alimentary liquid, and thus incorporated into drinks, or into lactic products such as yoghurts.

In a non-exhaustive way, the drinks can be, for example, fruit and/or vegetable juices.

The invention also relates to the use of the composition obtained for its dietary administration.

Finally, it should be reminded that the administration of the composition according to the invention advantageously permits to reduce the level of total cholesterol, and in particular the level of cholesterol related to low density lipoproteins (LDL), and/or to reduce the atherogenic index, and/or to increase the antioxidant capacities of blood plasma.

The administration of the composition according to the invention also permits to reduce the quantity of free radicals present in the organism, in particular by reducing the production of superoxide anion and/or reducing deposits of aortic lipid plaques.

The preferred dosage is between 0.5 and 0.8 grams of the composition per day when used as food supplement and 0.5 to 0.8 grams of the composition per liter when used in drinks. This dosage can be adapted so as to correspond to an administration between 10 and 25 mg, preferably approximately 21.5 mg, of composition per kilogram of body mass per day. As a matter of fact, 10 mg/kg body mass correspond approximately to the ORAC value content of a consumption of 5 fresh fruits and vegetables, and 21.5 mg/kg of body mass to the intake of 10 fresh fruits and vegetables, considering that the dosage can be modulated according to the desired intakes.

TABLE 1
Essential active substances

| Active substances | % of the composition |
|---|---|
| 1) Total polyphenols | 60-100% |
| Procyanidins (dimers, trimers) | 1-30% |
| Flavanols (monomers) | 5-50% |
| Anthocyanins | 0.1-10% |
| 2) Total carotenoids | 0.1-2% |
| Lycopene | 0.1-1% |
| Beta-carotene | 0.1-1% |
| 3) Vitamin C | 0.1-2% |
| 4) B-Type vitamins | 0.1-1.5% |
| Vitamin B1 (Thiamine) | 0.1-0.5% |
| Vitamin B6 (Pyridoxine) | 0.1-0.5% |
| Vitamin PP (Niacin) | 0.1-1% |

TABLE 2
Active substances detected in the composition according
to the invention used for the experiments

| Active substances | Content | |
|---|---|---|
| 1) Polyphenols | | |
| Dimers of Procyanidins B1, B2, B3 and B4 | 1.14 | g/100 g |
| Monomers of flavanols in the form of | | |
| monomeric catechins: | | |
| catechin | 0.55 | g/100 g |
| epicatechin | 3.08 | g/100 g |
| epicatechin-3-O-gallate | 4.10 | g/100 g |
| epigallocatechin | 4.17 | g/100 g |
| epigallocatechin-3-O-gallate | 21.33 | g/100 g |
| Anthocyanins | 0.6 | g/100 g |
| Gallic acid | 0.15 | g/100 g |
| 2) Carotenoids | | |
| Lycopene | 28 | mg/100 g |
| 3) Vitamin C | 4.92 | mg/100 g |

TABLE 3
Effects of the administration of the composition according
to the invention on the plasmatic concentrations of
lipids and the antioxidant capacity of blood plasma
in hamsters subjected to an atherogenie diet.

| | Control group | Experimental group |
|---|---|---|
| Initial weight (g) | 91.3 ± 2.1 | 86.7 ± 5.8 |
| Final weight (g) | 130.9 ± 9.7 | 129.6 ± 1.8 |
| Food intake (g/day) | 3.47 ± 0.90 | 3.49 ± 0.60 |

TABLE 4
Effects of the administration of the composition according
to the invention on the plasmatic concentrations of
lipids and the antioxidant capacity of blood plasma
in hamsters subjected to an atherogenic diet.

| | Control group | Experimental group |
|---|---|---|
| Total cholesterol (mmol/L) | 5.92 ± 0.17 | 5.00 ± 0.12 |
| Cholesterol related to | 3.81 ± 0.14 | 3.60 ± 0.09 |
| high density lipoproteins (mmol/L) | | |
| Cholesterol not related to | 2.18 ± 0.23 | 1.40 ± 0.13 |
| high density lipoproteins (mmol/L) | | |
| Atherogenic index | 1.59 ± 0.34 | 1.40 ± 0.18 |
| Antioxidant capacity of plasma | 1.29 ± 0.06 | 1.42 ± 0.10 |
| (mmol/L) | | |

TABLE 5
Essential vegetal species

| Vegetal species of | | % of the |
|---|---|---|
| the composition | Botanical name | composition |
| Fruit extracts | | |
| Grapes (red and white) | Vitis vinifera | 15-25% |
| Blueberry | Vaccinium myrtillus | 5-10% |
| Other extracts | | |
| Tomato | Solanum lycopersicum | 10-20% |
| Carrot | Daucus carota | 10-20% |
| Green tea | Camelia sinensis | 10-20% |

TABLE 6
Exhaustive list of vegetal species that can be included
in a composition according to the invention

| Vegetal species of | |
|---|---|
| the composition | Botanical name |
| Fruit extracts | |
| Grapes (red and white) | Vitis vinifera |
| Blueberry | Vaccinium myrtillus |
| Orange | Citrus aurantium dulcis |
| Grapefruit | Citrus grandis |
| Papaya | Carica papaya |
| Pineapple | Ananas sativus |
| Strawberry | Fragaria vesca |
| Apple | Purus malus |
| Apricot | Prunus armeniaca |
| Cherry | Prunus avium |
| Blackcurrant | Ribes nigrum |
| Other extracts | |
| Tomato | Solanum lycopersicum |
| Carrot | Daucus carota |
| Green tea | Camelia sinensis |
| Broccoli | Brassica oleracea |
| Green cabbage | Brassica oleracea |
| Onion | Allium cepa |
| Garlic | Allium sativum |
| Olive | Olea europaea |
| Wheat germs | germes de Triticum vulgare |
| Cucumber | Cucumis sativus |
| Asparagus | Asparagus officinalis |

1. Dietary composition, wherein the composition includes at least polyphenols and carotenoids.

2. Dietary composition according to claim 1, wherein the composition also includes vitamin C.

3. Dietary composition according to claim 1, wherein the composition also includes B-type vitamins.

4. Dietary composition according to claim 1, wherein the composition includes at least 60% of polyphenols by weight.

5. Dietary composition according to claim 1, wherein the composition includes 0.1 to 2% of carotenoids by weight.

6. Dietary composition according to claim 2, wherein the composition includes 0.1 to 2% vitamin C by weight.

7. Dietary composition according to claim 3, wherein the composition includes 0.1 to 1.5% B-type vitamins by weight.

8. Dietary composition according to claim 1, wherein the polyphenols are represented by at least 1 to 30% of procyanidins, 5 to 50% of flavanols and 0.1 to 10% of anthocyanins, the percentages being related to the total composition and the total polyphenols present attaining at least 60% by weight of the total composition.

9. Dietary composition according to claim 1, wherein the carotenoids are represented by at least lycopene and/or beta-carotene.

10. Dietary composition according to claim 3, wherein the B-type vitamins are represented by at least vitamin B1 and/or vitamin B6 and/or vitamin PP.

11. Dietary composition according to claim 1, wherein the composition is obtained from a mixture of vegetal species including at least red and/or white grapes (Vitis vinifera), blueberries (Vaccinium myrtillus), tomatoes (Solanum lycopersicum), carrots (Daucus carota), and green tea (Camelia sinensis).

12. Dietary composition according to claim 1, wherein the composition is obtained from a mixture of vegetal species including at least 15 to 25% by weight of red and/or white grapes (Vitis vinifera), 5 to 10% by weight of blueberries (Vaccinium myrtillus), 10 to 20% by weight of tomatoes (Solanum lycopersicum), 10 to 20% by weight of carrots (Daucus carota), and 10 to 20% by weight of green tea (Camelia sinensis).

13. Dietary composition according to claim 1, wherein the composition is obtained from a mixture including also one or more of the following vegetal species: orange (Citrus aurantium dulcis), grapefruit (Citrus grandis), papaya (Carica papaya), pineapple (Ananas sativus), strawberry (Fragaria vesca), apple (Purus malus), apricot (Prunus armeniaca), cherry (Prunus avium), blackcurrant (Ribes nigrum), broccoli (Brassica oleracea), green cabbage (Brassica oleracea), onion (Allium cepa), garlic (Allium sativum), olive (Olea europaea), wheat germs (germs of Triticum vulgare), cucumber (Cucumis sativus), and asparagus (Asparagus officinalis).

14. Dietary composition according to claim 1, wherein the composition is on its own or with additional additives, in the form of powder.

15. Dietary composition according to claim 1, wherein the composition is on its own or with addition additives, in the form of water-soluble powder.

16. Dietary composition according to claim 1, wherein the composition is on its own or with additional additives, in the form of encapsulated powder, in particular in the form of capsules.

17. Dietary composition according to claim 1, wherein the composition is on its own or with additional additives, in the form of compressed powder, in particular in the form of tablets or granules of all shapes.

18. Dietary composition according to claim 1, wherein the composition is on its own or with additional additives, in the form of powder put into sachets.

19. Dietary composition according to claim 1, wherein the composition is in the form of powder having an antioxidant capacity corresponding to at least 5,000 μmoles/gram of Trolox equivalents.

20. Method for obtaining a dietary composition according to claim 1, including the steps of extraction of active substances by maceration of ground materials to obtain a macerate, centrifugation of the obtained macerate, concentration of the active substances and drying by atomization, wherein one proceeds to the step of drying by atomization starting from a liquid phase, containing the active substances, having an antioxidant capacity between approximately 15,000 and 25,000 μmoles/gram of Trolox equivalents.

21. A food supplement comprising the dietary composition according to claim 1.

22. A method for enriching foods, in particular drinks such as fruit and/or vegetable juices as well as lactic products, comprising applying the dietary composition according to claim 1 to the foods.

23. A method for reducing the level of total cholesterol, in particular the level of cholesterol related to low density proteins (LDL), and/or the atherogenic index, and/or to increase the antioxidant capacities of blood plasma in a patient, comprising administering to a patient in need thereof a dietary amount of the dietary composition according to claim 1.

24. A method for reducing the quantity of free radicals present in an organism, in particular by reducing production of superoxide anions, and/or reducing deposits of aortic lipid plaques, comprising administering to the organism a dietary amount of the dietary composition according to claim 1.

25. A method for providing a dietary administration to a patient comprising administering the dietary composition according to claim 1 to a patient in need thereof according to a dosage between 10 and 25 mg, preferably approximately 21.5 mg, of the dietary composition per kilogram of body mass per day.
